(19) United States
(12) Patent Application Publication
(10) Pub. No.: US 2005/0192514 A1
(43) Pub. Date: Sep. 1, 2005

(54) Audiological treatment system and methods of using the same

(76) Inventors: Gerald Wayne Kearby, Loma Mar, CA (US); A. Robert Modeste, Redwood City, CA (US); Earl I. Levine, Palo Alto, CA (US); Walter John Greenleaf, Portola Valley, CA (US)

Correspondence Address: DAVID A. LEVINE, P. O. BOX 61180, PALO ALTO, CA, 94306-1180, US

(21) Appl. No.: 10/790,319

(22) Filed: Mar. 1, 2004

(51) Int. Cl. A61B 5/04; A61B 5/00; A61H 1/00; A61K 39/08

(52) U.S. Cl. 600/559; 600/544

(57) ABSTRACT

A system and method for treatment of neurological disorders with sound therapy is disclosed. The system can have a local PDA, a remote server, a physician's PC, and a database connected to the server. The PDA and PC can be networked with the server. The PC can initialize a treatment protocol stored at the server. The server can utilize the database and the protocol from the PC to come up with a recommended therapy. The recommended therapy can be selected by the PC and sent to the PDA. The PDA can then execute the selected therapy on the patient. The PDA can also record biometric and other feedback and send it to the server.

### BACKGROUND OF THE INVENTION

1. Field of the Invention

The present invention relates generally to devices and a system for providing diagnosis and therapy for audiological diseases and methods of using the same.

2. Description of the Related Art

Tinnitus is the sensation of noise that is caused by a bodily condition, such as disturbance of the auditory nerve or other neurological pathology. Absent of external auditory stimulus, tinnitus patients often hear one or more tones. The causes of tinnitus are believed to be numerous and still not quite fully understood. Typical tinnitus therapy includes drug therapy and/or sound or masking therapy, such as residual inhibition therapy.

Physicians use various testing procedures to determine the parameters of the tinnitus and the tones to be applied to the patient for specific types of therapy. A physician can perform an audible diagnostic test with qualitative patient feedback. Other testing procedures also include the use of expensive functional magnetic resonance imaging (FMRI), positron emission tomography (PET), electro-encephalograms (EEGs), Auditory event-related potential (ERP) and magnetic stimulation.

During sound therapy the patient is exposed to specific tones determined as a function of the tinnitus tones heard by the patient and any tones of which the patient has partial or total hearing loss. The tones used during sound therapy are intended to reduce the tinnitus symptoms. However, most effects from a single session of sound therapy are short lived, requiring patients to undergo repeated therapy sessions. In a few cases, fifteen minutes of residual inhibition therapy relieved tinnitus symptoms for a single day. Tinnitus symptoms vary significantly, not just from patient to patient, but also over time, with or without therapy, and over the course of therapy. Similarly, the therapeutic sounds (tones and volumes) that produce the best results vary from patient to patient, and the most effective therapeutic sounds often vary over the course of extended treatment.

The lack of effective sound therapies and flexible audiological platforms inhibits physicians from prescribing the most effective treatments for tinnitus. Furthermore, physicians must regularly test patients' progress to determine how to adjust the sound therapy. A large quantity of the physician's and patient's time and resources are used amending the prescribed therapies. This need for re-diagnosis also often leads to outdated, and thus imprecise, prescribed therapies, and also results in increased patient absenteeism for return visits to be re-evaluated and have sound therapies updated.

The ability to track patient treatment for sound therapy can be difficult, especially when the sound therapy occurs outside of a physician's office. The patient can be non-compliant by not following the sound treatment protocols. This lack of accurate tracking of the applied therapy impairs physicians' abilities to treat patients properly. The lack of accurate tracking also impedes health professionals attempting to create an accurate tinnitus model to use for predicting the most effective therapies and/or custom modification of an individual's therapy.

Therefore, there exists a need for a combined system to provide tinnitus therapy with frequent re-evaluation of the tinnitus profile. There also exists a need for an evaluation system reducing or obviating physician visits. There is also a need for a therapeutic and evaluation device that is easy to transport and use by the patient. Furthermore, there is a need for an accurate empirical model of tinnitus, such as a model that accurately predicts an effective sound therapy based on diagnostic inputs.

### BRIEF SUMMARY OF THE INVENTION

A system for treatment of an audiological deficiency is disclosed. The system has a remote device and a local device. The local device is communicating over a network to the remote device. The local device is configured to evaluate and treat the audiological deficiency.

The local device can treat the audiological deficiency according to a treatment protocol. The remote device can send a treatment protocol to the local device. The remote device can be a server.

The system can also have a database that is readable by the remote device. The database can have data regarding the audiological deficiency. The remote device can compute the treatment protocol with a computation. That computation can include use of the data.

The system can also have a physician's device communicating over a network to the remote device. The physician's device can control the treatment protocol.

Another system for treating an audiological deficiency is disclosed. The system has a remote device and a local device. The remote device is configured to access a database comprising data. The local device is configured to network with the remote device and create therapeutic audio. The therapeutic audio is determined from a function comprising the data.

A first device for application of a therapeutic protocol for audiological deficiencies is also disclosed. The device has a data transfer device and an acoustic transducer. The data transfer device is configured to communicate with a remote device. The data transfer device receives the therapeutic protocol from the remote device.

A method of treating a user having an audiological deficiency having symptoms is also disclosed. The method includes applying therapy for the symptoms using a portable device. The method also includes diagnosing the symptoms using the portable device.

The portable device can weigh less than about 1 pound. Diagnosing can include receiving user feedback during therapy. User feedback can include a biometric and/or a qualitative user response.

### BRIEF DESCRIPTION OF THE DRAWINGS

FIG. 1 illustrates an embodiment of an audiological treatment system.

FIG. 2 illustrates an embodiment of a local device.

FIG. 3 is a perspective view of an embodiment of a single earpiece.

FIG. 4 illustrates section A-A of the earpiece of FIG. 3.

FIG. 5 illustrates an embodiment of a method of audiological treatment.

FIG. 6 illustrates an embodiment of a method of initial audiological diagnosis.

FIG. 7 illustrates an embodiment of a method of determining if the patient is a suitable candidate for treatment.

FIG. 8 illustrates an embodiment of a method of sending the assessment data profile to the remote device.

FIG. 9 illustrates an embodiment of a method of sending data to produce and deliver the assessment report.

FIG. 10 illustrates an embodiment of a method of initial preparation of the local and remote devices.

FIG. 11 illustrates an embodiment of a method of the remote device producing an execution therapy report.

FIG. 12 illustrates an embodiment of a method of generating an initial recommended therapy report.

FIG. 13 illustrates an embodiment of a method of sending data to the database and the physician's device during initial patient assessment.

FIG. 14 illustrates an embodiment of a method of performing the prescribed evaluation and therapeutic use of the device.

FIG. 15 illustrates an embodiment of a method of the patient operating the local device.

FIG. 16 illustrates an embodiment of a method of synchronizing the local device and the remote device.

FIGS. 17 and 18 illustrate an embodiment of a method of data transfer during synchronization of the local device and the remote device.

FIG. 19 illustrates a method of sending data to the physician's device during or after the synchronization of the local device and the remote device.

FIG. 20 illustrates a method of sending data to the remote device and the database to update the therapy.

FIG. 21 illustrates an embodiment of a method of the remote device analyzing the treatment data.

### DETAILED DESCRIPTION

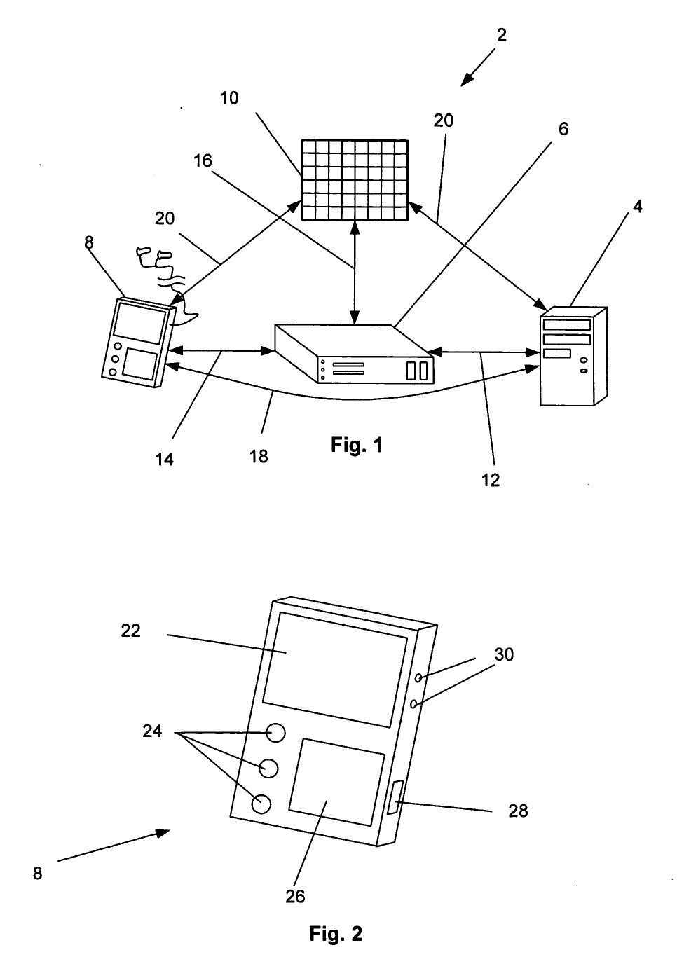

FIG. 1 illustrates a neurological treatment system 2system 2. The treatment herein can include augmentation and/or diagnosis and/or therapy. The condition that can be treated can be any neurological process amenable to treatment or augmentation by sound, for example otological or audiological disorders such as tinnitus or other pathologies where retraining of the auditory cortex using auditory stimulus and/or training protocols to improve function is possible. Other examples of treatment of audiological conditions include refining or training substantially physiologically normal hearing, stuttering, autism or combinations thereof.

The system 2system 2 can have a physician's device 4, a remote device 6, a local device 8 and a database 10. The physician's device 4 can be configured to communicate, shown by arrows 12, with the remote device 6. The remote device 6 can be configured to communicate with the local device 8, shown by arrows 14. The remote device 6 can be configured to communicate, shown by arrows 16, with the database 10. The physician's device 4 can be configured to communicate directly, shown by arrows 18, with the local device 8. The database 10 can be configured to communicate directly, shown by arrows 20, with the local device 8 and/or the physician's device 4.

The physician's device 4, the remote device 6 and the local device 8 can be, for example, laptop or desktop personal computers (PCs), personal data assistants (PDAs), network servers, portable (e.g., cellular, cordless) telephones, portable audio players and recorders (e.g., mp3 players, voice recorders), car or home audio equipment, or combinations thereof. The physician's device 4, the remote device 6 and the local device 8 can be processors connected on the same circuit board, components of the same processor, or combinations thereof and/or combinations with the examples supra. The physician's device 4, the remote device 6 and the local device 8, or any combination thereof, can be a single device of any example listed supra, for example a single PC or a single, integrated processor.

The database 10 can be structured file formats, relational (e.g., Structured Query Language types, such as SQL, SQL1 and SQL2), object-oriented (e.g., Object Data Management Group standard types, such as ODMG-1.0 and ODMG-2.0), object-relational (e.g., SQL3), or multiple databases of one or multiple types. The database 10 can be a single set of data. The database 10 can be or comprise one or more functions. The database 10 can be stored on the remote device 6. The database 10 can be stored other than on the remote device 6.

The communications 12, 14, 16, 18 and 20 can be via hardwiring (e.g., between two processors or integrated circuit devices on a circuit board), transferable media (e.g., CD, floppy disk, removable flash memory device, SIM card, a smart card, USB based mass storage device), networked connection (e.g., over the internet, Ethernet (IEEE 802.3), universal serial bus (USB), Firewire (IEEE 1394), 802.11 (wireless LAN), Bluetooth, cellular communication modem), direct point-to-point connection (e.g., serial port (RS-232, RS-485), parallel port (IEEE 1284), Fiber Channel, IRDA infrared data port, modem, radio such as 900 MHz RF or FM signal) or combinations thereof. The communications 12, 14, 16, 18 and 20 can be constant or sporadic.

The physician's device 4 can have local memory. The memory can be non-volatile, for example a hard drive or non-volatile semiconductor memory (e.g., flash, ferromagnetic). A copy of all or part of the database 10 can be on the local memory of the physician's device 4. The physician's device 4 can be configured to communicate with the database 10 through the remote device 6.

The remote device 6 can be configured to transfer data to and from the physician's device 4, the local device 8 and/or the database 10. The data transfer can be through a port (e.g., USB, Firewire, serial, parallel, Ethernet), a media player and/or recorder (e.g., CD drive, floppy disk drive, smart card reader/writer, SIM card, flash memory card reader/writer (e.g., Compact Flash, SD, Memory Stick, Smart Media, MMC), USB based mass storage device), a radio (e.g., Bluetooth, 802.11, cellular or cordless telephone, or radio operating at frequencies and modulations such as 900 Mhz or commercial FM signals) or combinations thereof.

Data stored in the database 10 can include all or any combination of the data found in patient profiles, profile assessment data, relevant assessment data, execution therapy reports, recommended therapy reports, physician's therapy reports, executed session reports and analyzed session reports, several described infra. The reports can be compressed and decompressed and/or encrypted and decrypted at any point during the methods described herein. The reports can be script, XML, binary, executable object, text files and composites of combinations thereof.

FIG. 2 illustrates the local device 8. The local device 8 can be portable. The local device can be less than about 0.9 kg (2 lbs.), more narrowly less than about 0.5 kg (1 lbs.), yet more narrowly less than about 0.2 kg (0.4 lbs.), for example about 0.17 kg (0.37 lbs.). For example, the local device 8 can be a graphic user interface (GUI) operating system (OS) PDA (e.g., the Yopy 500 from G.Mate, Inc., Kyounggi-Do, Korea).

The local device 8 can receive power from an external power source, for example a substantially unlimited power supply such as a public electric utility. The local device 8 can have a local power source. The local power source can be one or more batteries, for example rechargeable batteries, photovoltaic transducers, or fuel cells (e.g., hydrocarbon cells such as methanol cells, hydrogen cells). The local device 8 can be configured to optimize power consumption for audio output.

Power consumption can be reduced by placing sub-systems that are not in use into a low power state (e.g., sleep). Power consumption can be reduced by placing sub-systems that are not in use into a no power state (e.g., off). Power consumption can be reduced by dynamically changing the frequency of the clock governing one or more sub-systems.

Power consumption can be reduced by the inclusion of a specialized sound generation/playback integrated circuit. The specialized sound generation/playback integrated circuit can generate the therapeutic sounds through direct generation of the therapeutic sounds and/or can playback stored therapeutic sound. Power consumption of the specialized sound generation/playback integrated circuit can be substantially lower than other processing elements within the local device. During operation of the specialized sound generation/playback integrated circuit the other processing elements of the device can be placed into a low power or no power state. The power consumption reduction methods supra can be used individually or in any combination.

The local device 8 can have local memory, for example flash memory. The amount of local memory can be from about 64 kB to about 128 MB, more narrowly from about 1 MB to about 32 MB, yet more narrowly from about 4 MB to about 16 MB. The local device 8 can have a processor. The processor can have, for example, a clock speed equal to or greater than about 16 MHz, more narrowly equal to or greater than about 66 MHz. The local memory can be a portion of a larger memory device. The local device 8 can have random access memory (RAM) for the treatment available to the processor. The amount of RAM for the treatment can be equal to or greater than about 4 Mb, more narrowly equal to or greater than about 32 Mb. The RAM for the treatment can be a portion of a larger a quantity of RAM available to the processor. The local device 8 can have a real-time clock. The clock, for example a real-time clock, can be used to time stamp (i.e., couple with temporal data) any data within the local device. Data that can be time stamped can include data from any reports or transmission of any report or data, such as for reports pertaining to therapy sessions and conditions. Time stamp data can include relative or absolute time data, such as year, calendar date, time of day, time zone, length of operation data and combinations thereof.

The local device 8 can have a visual screen 22. The visual screen 22 can be a visual output and/or input, for example a transparent touch-pad in front of a display. The visual output can be a liquid crystal display (LCD) including an organic LCD, cathode ray tube, plasma screen or combinations thereof. The local device 8 can have user controls 24. The user controls 24 can be knobs, switches, buttons, slides, touchpads, keyboards, trackballs, mice, joysticks or combinations thereof. The user controls 24 can be configured to control volume, provide feedback (e.g., qualitative ranking, such as a numerical score, text or speech messages to physician), control the treatment, change treatment modes, set local device 8 parameters (e.g., day, month, year, sensor input parameters, default settings), turn local device 8 on or off, initiate communication and or synchronization with remote device 6, initiate communication and or synchronization with the physician's device 4 or combinations thereof.

The local device 8 can have one or more external transducers 26. The external transducers 26 can be audio transducers, for example speakers and/or microphones. The external transducers 26 can sense ambient conditions (e.g., noise/sound, temperature, humidity, light, galvanic skin response, heart rate, respiration, EEG, auditory event-related potentials (ERP)) and/or be used to record verbal notes. The external transducers 26 can emit sound. The local device 8 can store in the local device's memory signals detected by the sensors and transducers of the local device 8. The sensor and transducer data can be stored with time stamp data.

The local device 8 can have a data transfer device 28. The data transfer device 28 can be a port (e.g., USB, Firewire, serial, parallel, Ethernet), a transferable storage media reader/writer (e.g., CD drive, floppy disk drive, hard disk drive, smart card, SIM card, flash memory card (e.g., Compact Flash, SD, Memory Stick, Smart Media, MMC), USB based mass storage device), a radio (e.g., Bluetooth, 802.11, cellular or cordless telephone, or radio operating at frequencies and modulations such as 900 Mhz or commercial FM signal) or combinations thereof. The data transfer device 28 can facilitate communication with the remote device 6.

The local device 8 can have one or more local device connectors 30. The local device connectors 30 can be plugs and/or outlets known to one having ordinary skill in the art. The local device connectors 30 can be cords extending from the local device 8. The cords can terminate attached to plugs and/or outlets known to one having ordinary skill in the art. The local device connectors 30 can be media players/recorders (e.g., CD drive, floppy disk drive, hard drive, smart card reader, SIM card, flash memory card, USB based mass storage device). The local device connectors 30 can be radio (e.g., Bluetooth, 802.11, radio, cordless or cellular telephone).

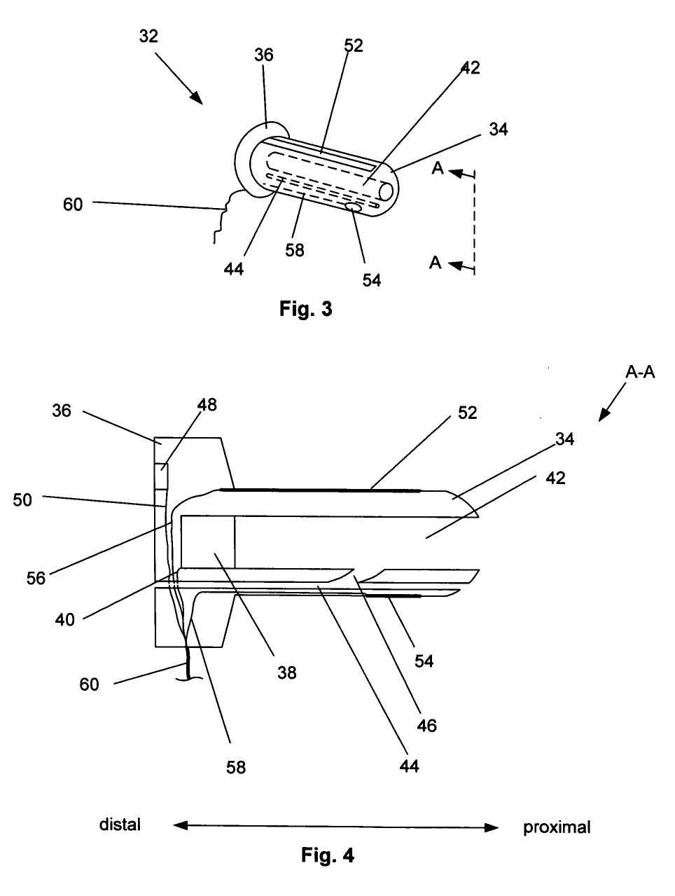

The local device 8 can have one, two or more earpieces. The local device connectors 30 can facilitate communication with the earpiece 32. FIG. 3 illustrates the earpiece 32 that can have a probe 34 attached to a retention element 36. FIG. 4 illustrates cross-section A-A of the earpiece of FIG. 3. The probe 34 can be shaped to fit intra-aurally. The earpiece 32 can be shaped to fit entirely supra-aurally. All or part of the retention element 36 can be shaped to fit in the intertragic notch. The retention element 36 can be shaped to fit circumaurally. The retention element 36 can be padded. The probe 34 and/or the retention element 36 can be molded to fit the specific ear canal and intertragic notch for a specific patient.

The earpiece 32 can have a therapy transducer 38. The therapy transducer 38 can be an acoustic transducer, for example a headphone speaker. A therapy lead 40 can extend from the therapy transducer 38.

An acoustic channel 42 can extend from the therapy transducer 38 to the proximal end of the probe 34 The earpiece 32 can have an ambient channel 44 from the distal end of the earpiece 32 to the proximal end of the earpiece 32. The ambient channel 44 can merge, as shown at 46, with the acoustic channel 42. The ambient channel 44 can improve transmission of ambient sound, humidity and temperature through the earpiece 32. The ambient channel 44 can be a channel from the distal end to the outside and/or proximal end of the earpiece 32.

The earpiece 32 can have one or more ambient conditions sensors 48. The ambient conditions sensors 48 can sense ambient sound frequency and/or amplitude, temperature, light frequency and/or amplitude, humidity or combinations thereof. An ambient lead 50 can extend from the ambient conditions sensor 48.

The earpiece 32 can have one or more biometric sensor strips 52 and/or biometric sensor pads 54. The biometric sensors 52 and 54 can be configured to sense body temperature, pulse (i.e., heart rate), perspiration (e.g., by galvanic skin response or electrodermal response), diastolic, systolic or average blood pressure, electrocardiogram (EKG), brain signals (e.g., EEG, such as EEG used to determine sensory threshold audio levels, auditory event-related potentials (ERP)), hematocrit, respiration, movement and/or other measures of activity level, blood oxygen saturation and combinations thereof. The biometric sensors 52 and 54 can be electrodes, pressure transducers, bimetallic or thermister temperature sensors, optical biometric sensors, or any combination thereof. An example of optical biometric sensors is taught in U.S. Pat. No. 6,556,852 to Schulze et al., which is hereby incorporated by reference in its entirety. A strip lead 56 can extend from the biometric sensor strip 52. A pad lead 58 can extend from the biometric sensor pad 54.

The leads 40, 50, 56 and 58 can each be one or more wires. The leads 40, 50, 56 and 58 can carry power and signals to and from their respective transducer 38 and sensors 48, 52 and 54.

The leads 40, 50, 56 and 58 can attach to an earpiece connector 60. The earpiece connector 60 can be one or more cords extending from the earpiece 32. The cords can terminate attached to plugs and/or outlets (not shown) known to one having ordinary skill in the art. The earpiece connector 60 can be a plug and/or an outlet known to one having ordinary skill in the art. The earpiece connector 60 can be a media player/recorder (e.g., CD drive, flash memory card, SIM card, smart card reader). The earpiece connector 60 can be a processor and/or a radio (e.g., Bluetooth, 802.11, cellular telephone, radio). The earpiece connector 60 can connect to the local device connector 30 during use.

### METHODS OF TREATMENT

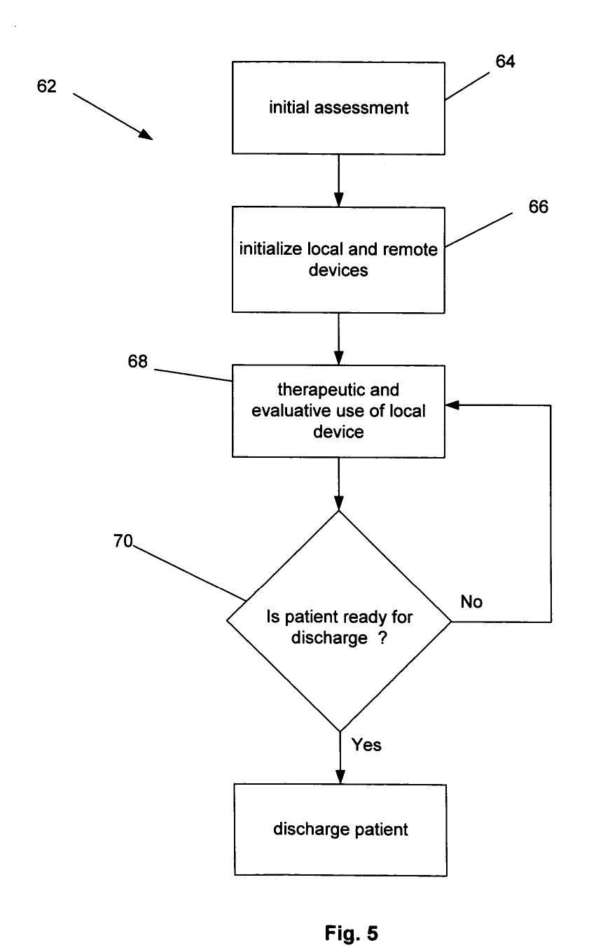

FIG. 5 illustrates a method of treatment 62, such as a neurological or audiological treatment. (For exemplary clarity the treatment 62 is referred to hereafter, non-limitingly, as the audiological treatment 62.) An initial assessment 64 of an audiological disorder, such as tinnitus, can be made, for example by a physician during a visit with a patient. The local and remote devices 8 and 6 can then be initialized 66. The local device 8 can then be used for evaluation and/or therapy 68. After use 68, if the patient is not ready to be discharged from therapy, the query as shown by 70, using the local device 8 for diagnosis or re-evaluation and therapy 68 can be repeated. After use 68, if the patient is ready to be discharged from therapy, the patient can be discharged from the treatment.

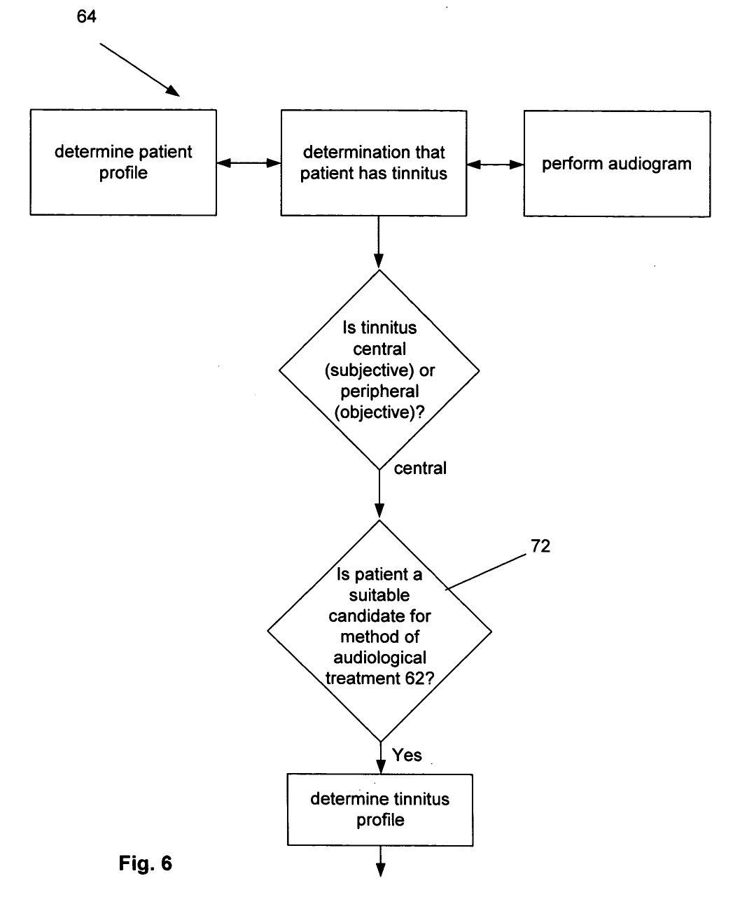

FIG. 6 illustrates making the initial assessment 64 of an audiological disorder. The physician can determine that the patient has the audiological disorder, such as tinnitus. (For exemplary clarity the audiological disorder is referred to hereafter, non-limitingly, as tinnitus.) The physician can perform an audiogram on the patient before or after the determination of tinnitus. The physician can determine the patient profile (e.g., gender, age, career, existing and cured health problems, allergies, biometrics such as blood pressure and temperature, stress, exertion, tension, presence of noise, rest, insurance company and policy, length of time of affliction, precipitating event), for example, from the combination of a pre-existing file and/or an interview and/or exam. The physician can determine whether the tinnitus is central (i.e., subjective) or peripheral (i.e., objective). If the tinnitus is central (or the other neurological disorder can be corrected by sound therapy), the patient can be analyzed, as shown by 72, to determine if the patient is a suitable candidate for the method of audiological treatment 62. If the patient is a suitable candidate for therapy, the audiological treatment 62 can proceed to the initialization 66 of the local and remote devices 8 and 6.

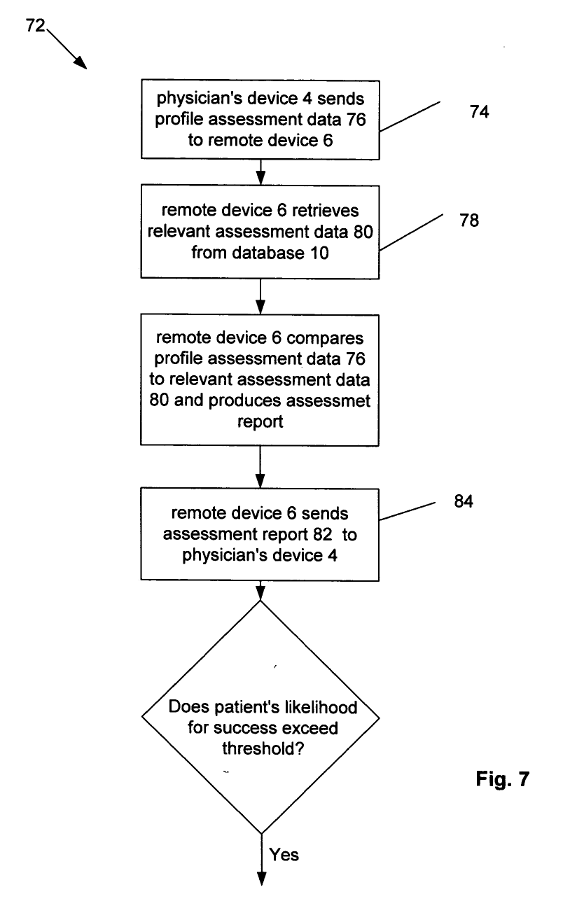

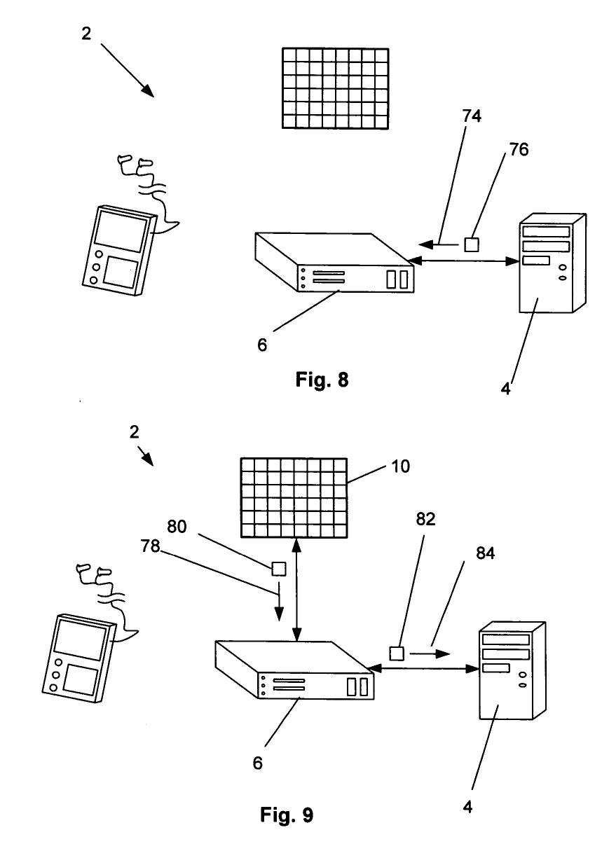

The patient's tinnitus profile can be determined after the physician has determined that the patient has tinnitus. The tinnitus profile can include the symptom tones and the respective amplitudes for each tone. The tinnitus profile can include tones for which the patient has partial or total hearing loss, the degree of hearing loss at each of the tones, an objectively and/or subjectively determined impairment score or combinations thereof. FIG. 7 illustrates, as shown by 72, determining whether the patient is a suitable candidate for treatment by the method of treatment 62.

As shown by 74 in FIG. 8, the physician's device 4 can send profile assessment data 76 to the remote device 6. The profile assessment data 76 can be all or part of the patient profile, tinnitus profile, additional hearing tests or any combination thereof.

As shown by 78 in FIG. 9, the remote device 6 can retrieve relevant assessment data 80 from the database 10. The relevant assessment data 80 can include data from patients with similar profile assessment data 76. The relevant assessment data 80 can include profile assessment data 76, treatment efficacy, treatment protocols, summaries of any of the aforementioned data (e.g., as single or multi-dimensional indices) and combinations thereof. The remote device 6 can compare the profile assessment data 76 to the relevant assessment data 80. This comparison can, for example, determine the optimal treatment protocol for the patient. The comparison can be performed with static and/or modeling techniques (e.g., data-mining).

For example, the profile assessment data 76 can be compared to the relevant assessment data 80 and the best matches of pretreatment conditions can be determined therefrom. Of the successful matches, the treatment protocols used to generate successful outcomes (e.g., results above a threshold level) can be assessed and averaged. This average can be used to derive an assessment report.

The remote device 6 can then produce the assessment report 82 and send the assessment report 82 to the physician's device 4, as shown by 84 in FIG. 9. The remote device 6 can send the assessment report 82 to a third party, for example, an insurance company. The assessment report 82 can be printed and sent as a hard copy, or sent as a file via an e-mail, file transfer protocol (FTP), hypertext transfer protocol (HTTP), HTTP secure (HTTPS) or combinations thereof. The assessment report 82 can be encrypted. The assessment report can be compressed.

The assessment report 82 can include the assessment data, a likelihood of patient success, a threshold success level for the patient, a recommendation regarding whether the patient's likelihood exceeds the patient's threshold success level, a prognosis, an initial recommended therapy report, graphs of all collected data comparing the patient to similar patients, case examples of similarly assessed patients or combinations thereof. Therapy reports can include a protocol or prescription for administering sound therapy sessions. The protocol can include one or more sounds, such as therapeutic audio. The sounds can include one or more tones, gains and/or amplitudes for each tone, one or more noise profiles (e.g., the shape of the power spectrum), music, mechanical representation of the determined audio treatment information, overall gains and/or amplitudes for each noise profile, other sounds (e.g., buzzes, swirling, modulated tones, pulses) and their respective overall gains and/or amplitudes, a therapy schedule, recommended re-evaluation dates and/or times, and combinations thereof.

The therapy schedule can include when (e.g., dates and/or times) each tone and/or noise is to be played, how long each tone and/or noise is to be played, instructions for the patient and/or the system 2system 2 regarding what to do if a therapy is missed.

The therapy report can be a script, XML, binary, executable object, text file and composites of combinations thereof. The therapy report can be encrypted. The therapy report can be compressed.

The threshold success level for the patient can be assigned a value by the patient's insurance company. The threshold success level can be assigned a value based on normative database averages. The threshold success level can be assigned a value by the physician. The physician can then determine whether the patient's likelihood for success exceeds the threshold success level for the patient. The physician can overrule the remote device's recommendation of whether the patient's likelihood for success exceeds the patient's threshold success level. If the physician determines to continue with the method of audiological treatment 62, the local and remote devices 8 and 6 can be initialized 66.

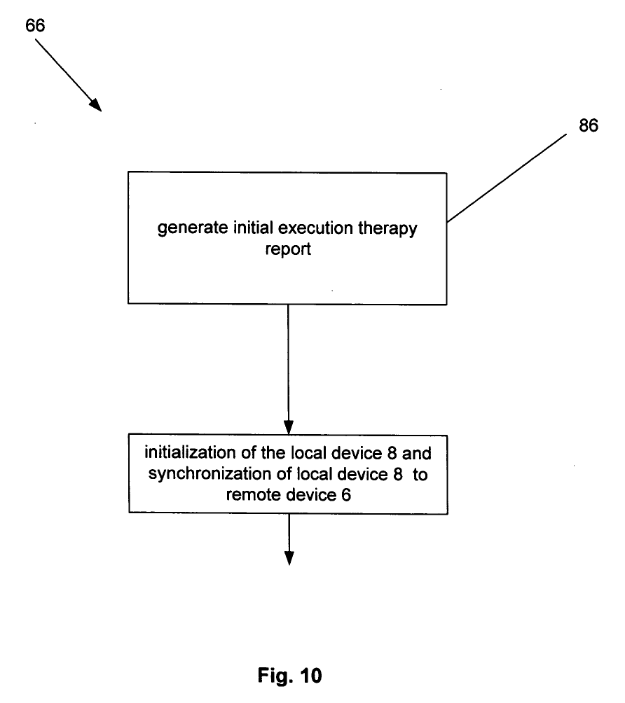

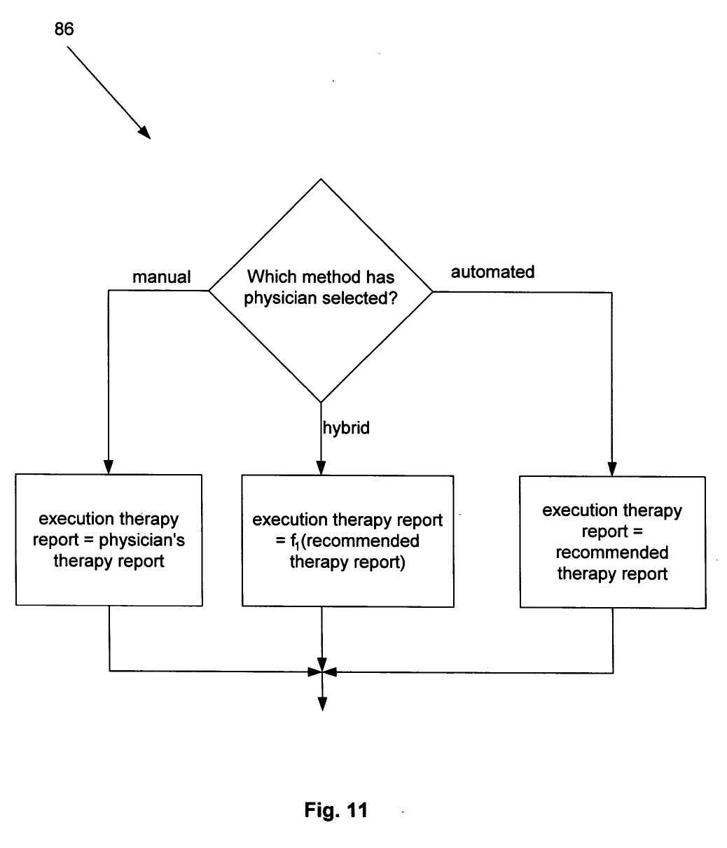

FIG. 10 illustrates the initialization 66 of the local and remote devices 8 and 6. An initial execution therapy report can be generated, as shown by 86, for example, by using the recommended therapy report from the assessment report 82 and/or using a physician's therapy report from the physician. The execution therapy report can contain the therapy report that will be executed by the local device 8.

The physician's therapy report can include the physician's selection as to present and future methods of generating the execution therapy report. The execution therapy report can be entirely copied from the physician's therapy report (i.e., a manual selection), entirely copied from the recommended therapy report (i.e., an automated selection), or generated by the remote device 6 as a function of the recommended therapy report and the physician's therapy report (i.e., a hybrid selection).

FIG. 11 illustrates a method for generating the initial execution therapy report. If the physician's therapy report has a manual selection, the execution therapy report can be copied from the physician's therapy report.

If the physician's therapy report has an automated or default selection, the execution therapy report can be copied from the recommended therapy report.

If the physician's therapy report has a hybrid selection, the physician's therapy report and the recommended therapy report can be processed by a function (f₁) that results in the execution therapy report. That function can be generated, by the physician modifying any of the data in the recommended therapy report. For example, the physician can modify the recommended therapy report to include additional scheduled treatment sessions.

The local device 8 can be initialized by deleting prior patient information from the memory of the local device 8 and restoring the settings to a default state. The local device 8 can then be synchronized to the remote device 6 as described infra.

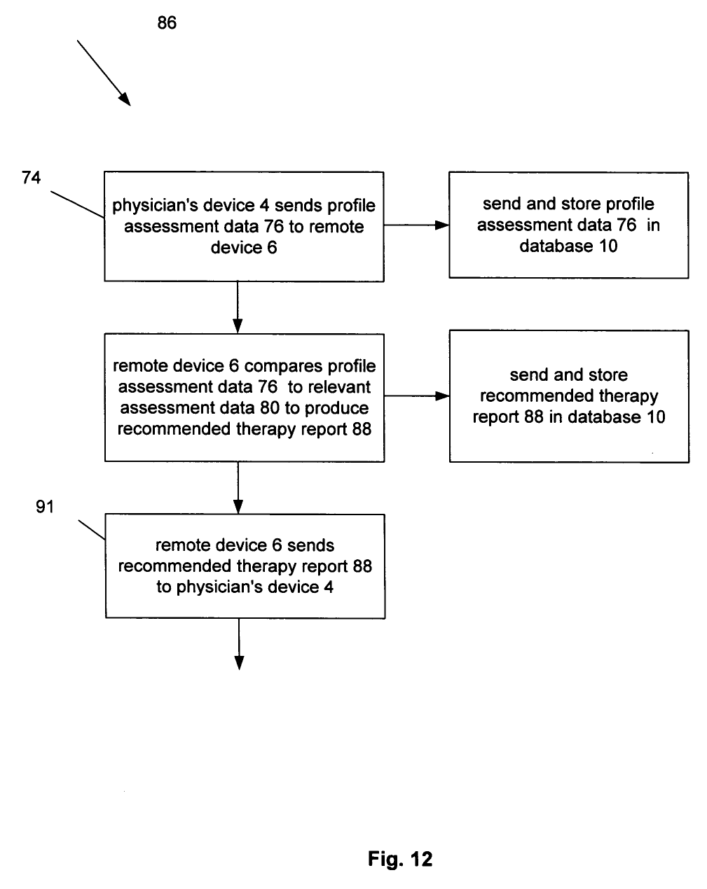

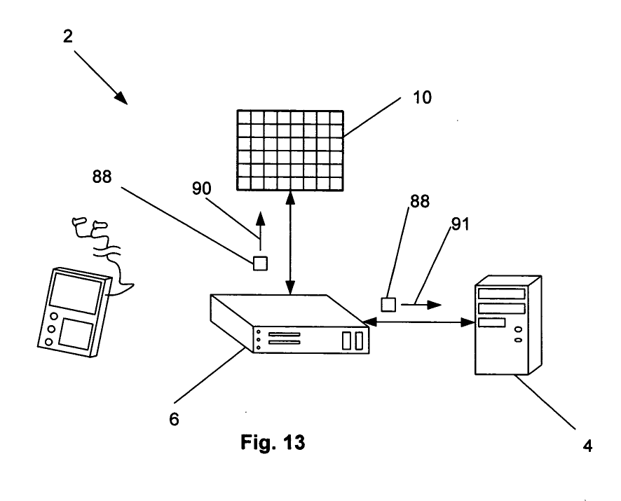

FIG. 12 illustrates generating the recommended therapy report The physician's device 4 can send the profile assessment data 76 to the remote device 6, as shown by 74 in FIG. 8. The remote device 6 can send and store (not shown) the profile assessment data 76 in the database 10.

The remote device 6 can then compare the profile assessment data 76 to the relevant assessment data 80 to produce a recommended therapy report. For example, the remote device 6 can identify that the volume level for the perceived tinnitus tone has decreased as a result of treatment, and consequently modify the volume in the recommended therapy report.

The remote device 6 can send and store the initial recommended therapy report 88 in the database 10, as shown by 90 in FIG. 13. The remote device 6 can send the initial recommended therapy report 88 to the physician's device 4. The remote device 6 can send the initial recommended therapy report 88 to a third party, for example, an insurance company or health monitoring organization.

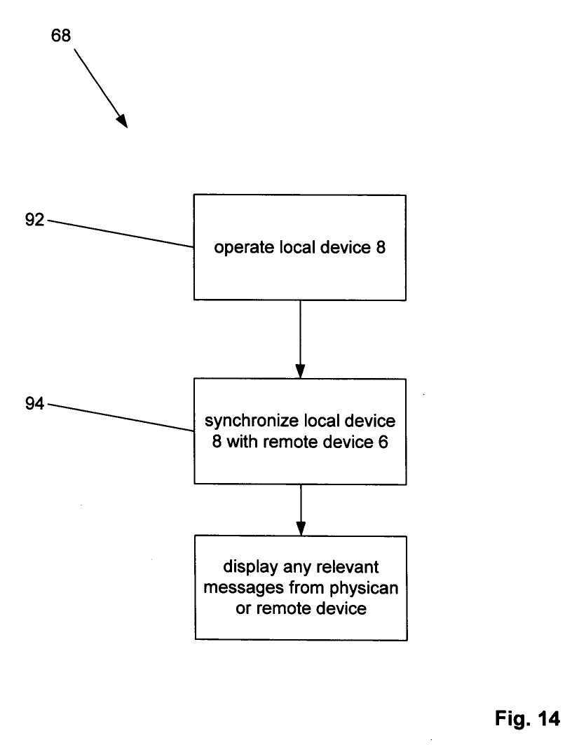

FIG. 14 illustrates, as shown by 68, evaluation and therapeutic use of the local device 8. The local device 8 can be operated, shown by 92, for example by the patient on the patient. The local device 8 can then be synchronized, shown by 94, with the remote device 6. The local device 8 can display or play any messages from the remote device or the physician for the patient to read or hear.

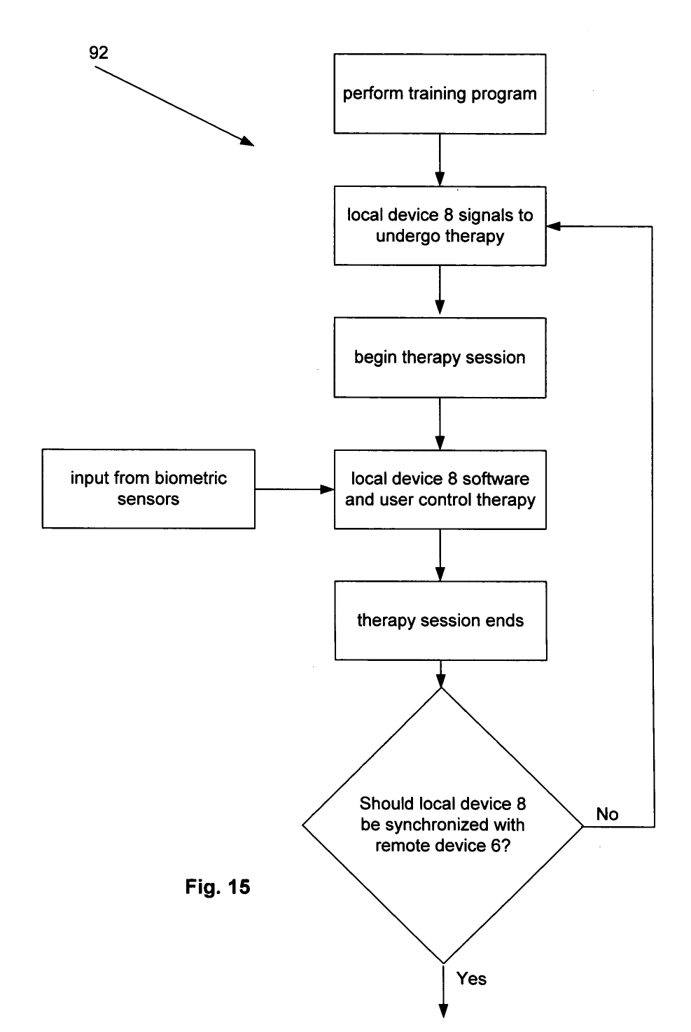

FIG. 15 illustrates operation of the local device 8. A training program on the local device 8 can be performed, for example by the patient. The training program can orient and teach the user operation of the local device 8. The training program can teach the user the importance of proper use of the system 2.

The training program can be skipped by the user automatically or by the local device 8, for example after the first use. The ability to skip the training program can be inhibited by the physician as part of the execution therapy report.

When the therapy schedule of the execution therapy report calls for therapy, the local device 8 can signal the patient to undergo therapy. The signal can be audible, visual, vibratory or a combination thereof. The patient can then apply the local device 8. Application of the local device 8 can include placing the speaker close enough to be heard at the desired volume and/or wearing the earpiece. The sound therapy session can then begin. The patient can receive the sound therapy by listening to the sound therapy session. The listening can include listening over the on-board speaker (i.e., the external transducer 26) and/or listening through the earpieces 32 or other auxiliary speakers.

While delivering the sound therapy session, the local device 8 can be controlled by the software. The local device 8 can run the sound therapy session (e.g., schedule, tones, gain) as prescribed by the execution therapy report. The local device's software can adjust the volume based on the ambient noise level. The volume can be adjusted so that emitted sound can be appropriately perceived by the patient given the ambient noise level.

The local device's software can apply feedback from biometric sensors 52 and 54 to the local device 8. For example, the patient's heart rate signal can be used as part of a biofeedback system to relax the patient while listening to the emitted sound.

The biometric sensors 52 and 54 can be internal or external to the local device 8. The local device 8 can use the biometric values to determine the efficacy of the treatment and adjust the treatment during or between sessions based on the efficacy. The biometrics can be sensed and recorded by the local device 8. The biometrics can be constantly or occasionally sensed and displayed to the user during use of the local device 8. The user can be informed of the efficacy of the treatment. The user can attempt to consciously control the biometrics (e.g., slow the heart rate by consciously calming).

The local device's software can play audio and/or visual messages from the physician's device 4 stored in the execution therapy report.

The patient can control the therapy. The patient can adjust the therapeutic amplitudes/gain and tones, for example with a mixer. The patient can also select a background sound to be delivered with the therapy session. Background sounds include music, nature sounds, vocals and combinations thereof. The user can select predefined modes for the local device 8. For example, the user can select a mode for when the user is sleeping (e.g., this mode can automatically reduce the sound amplitude after a given time has expired), a driving mode (e.g., this mode can play ambient noise with the sound therapy session, or set a maximum volume), a noisy mode, a quiet mode, an off mode or combinations thereof. The patient can remove the local device 8 from audible range, effectively stopping therapy. The local device 8 can record the therapy stoppage in the session report.

Patient feedback can be sent to the local device 8 during or after a therapy session. For example, the patient can provide a qualitative rating of the therapy (e.g., thumbs-up/thumbs-down, or on a ten-point scale), record verbal or text notes regarding the therapy into the memory of the local device 8 or combinations thereof. Any biometrics (e.g., as measured by the local device 8 or by another device) can be entered into memory of the local device 8, manually entered through the local device 8 if necessary. The feedback, biometric and/or non-biometric, can be time and date stamped.

As FIG. 15 illustrates, when the sound therapy session ends, the local device 8 can be synchronized with the remote device 6, as shown by 94. The local device 8 can signal that it should be synchronized with the remote device 6. The user can also synchronize the local device 8 without a signal to synchronize.

During use of the local device 8, the local device 8 can perform a sensory threshold test. The sensory threshold test can be initiated by the user or the local device 8. The sensory threshold test can be performed on a frequency (e.g., before every therapy session, every morning, once per week) assigned by the execution therapy report.

During the sensory threshold test, the local device 8 can emit the user's tinnitus tones to the user. The local device 8 can then adjust the amplitude of the produced tones (e.g., trying higher and lower amplitudes, using the method of limits). The user can send feedback to the local device 8 regarding the user's ability to match the amplitudes of the user's natural tinnitus tones to the amplitudes of the local device-generated tones. The local device 8 can then store the resulting amplitudes in the executed session report. The user and/or the local device 8 can adjust the local device-generated tones individually (e.g., with a manually-controlled mixer on the local device and/or to account for ambient sounds).

After a therapy session ends, the local device 8 can produce an executed session report. The executed session report can include all executed session data that has occurred since the last synchronization 94 between the local device 8 and the remote device 6. The session data can include the usage (e.g., number of times used, length of time used, time of day used, date used, volume at which it was used), patient feedback (e.g., qualitative rating of the therapy, verbal or text notes, biometric feedback or combinations thereof), prior therapy reports, including the immediately prior therapy report. Subjective feedback from the user can be solicited by the local device 8 by use of interactive entertainment (e.g., a game).

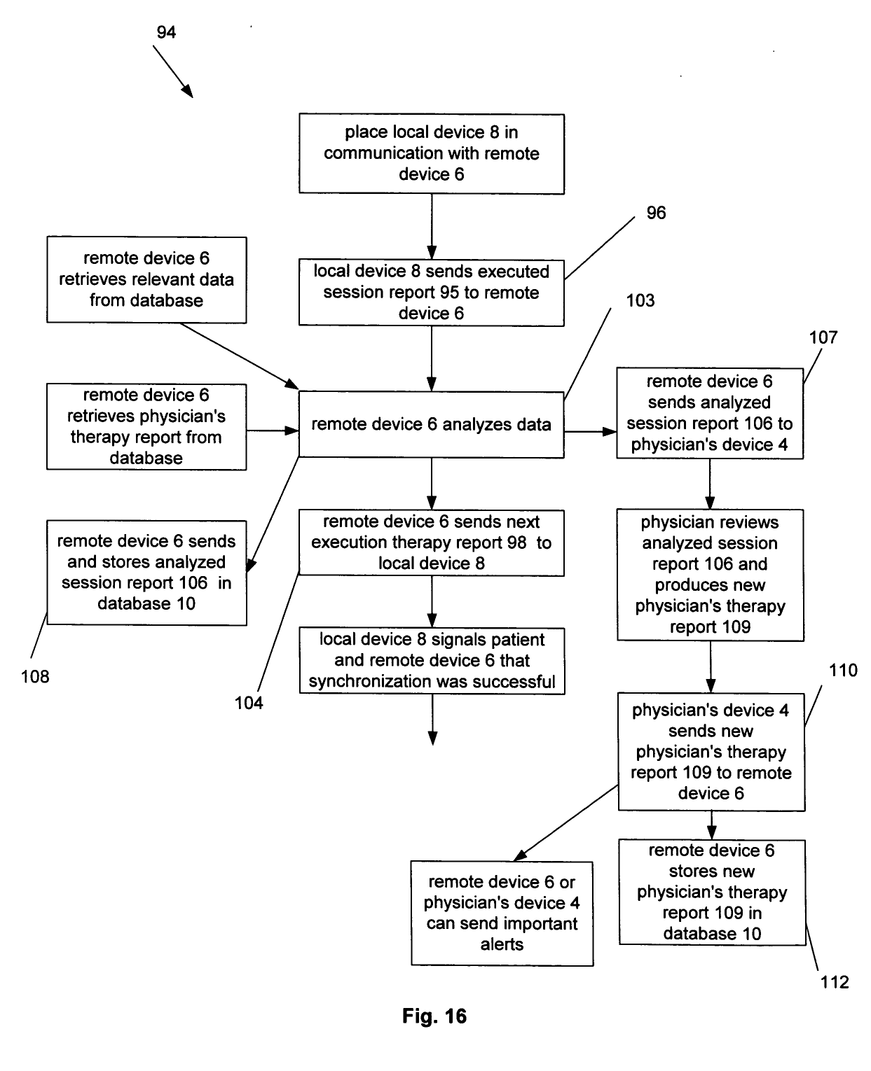

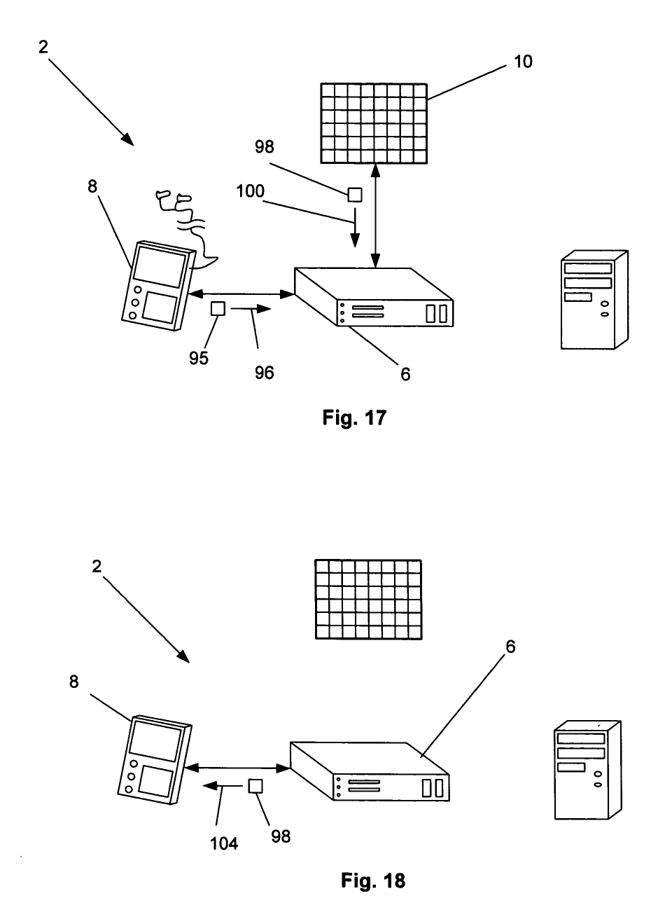

FIG. 16 illustrates that the local device 8 can be placed in communication with the remote device 6. The local device 8 can then send the executed session report 95 to the remote device 6, as shown by 96 in FIG. 17. The executed session report 95 can be encrypted. The executed session report 95 can be compressed.

The remote device 6 can retrieve from the database 10 the execution therapy report 98 to be executed next by the local device 8, as shown by 100 in FIG. 17. The remote device 6 can analyze the executed session report 95, the to-be-executed-next execution therapy report 98, and data from the database (including data from the patient). The remote device 6 can produce an analyzed session report 106.

Statistical methods and algorithms can be used to compare expected patient progress with actual patient progress. Changes in the patient protocol can be generated, at least in-part, based on this analysis. Changes can include, for example, lengthening or shortening the amount of treatment time, changes in tone volume, recommendation for reevaluation.

The analyzed session report 106 can include the session data, an analysis including a new recommended therapy report. The new recommended therapy report can be modified based, at least in-part, on the analysis of session data,. For example, if the patient's progress is not as predicted or expected, the amplitude of the treatment tone can be increased, the duration of the treatment can be increased, a new treatment may be added or combinations thereof.

As shown by 103 in FIG. 16, the remote device 6 can analyze the recommended therapy report, the physician's therapy report and the analyzed session report and produce a new execution therapy report. The new execution therapy report can include the same categories of data as the initial execution therapy report.

The remote device 6 can send the to-be-executed-next execution therapy report 98 to the local device 8, as shown by 104 in FIG. 18. The local device 8 can signal to the patient and the remote device 6 that synchronization was successful. The success of the synchronization can be logged in the analyzed session report. The local device 8 can display any urgent messages.

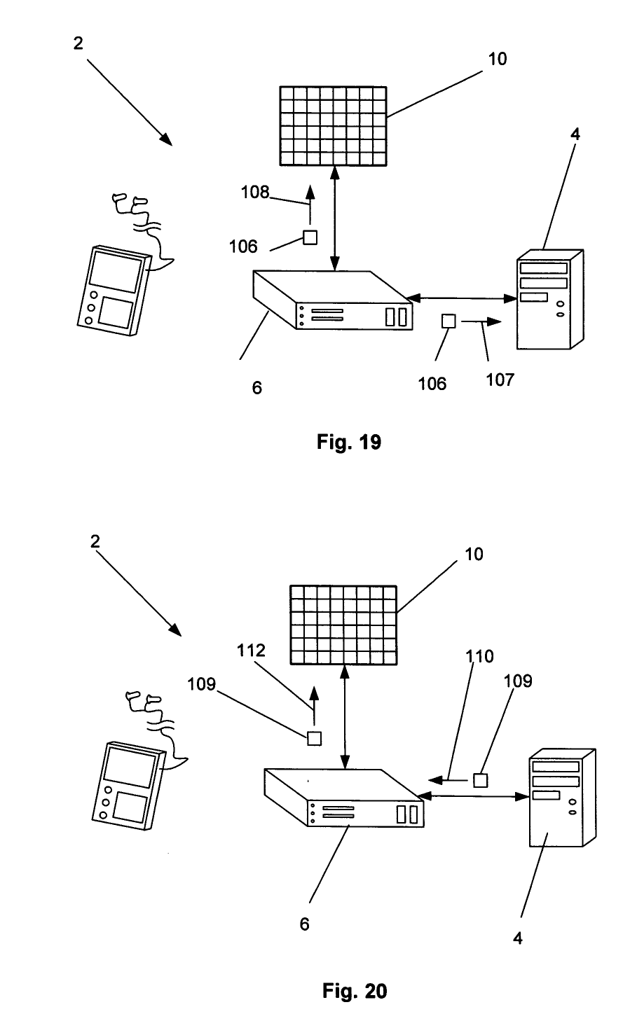

The remote device 6 can send and store the analyzed session report 106 in the database 10, as shown by 108 in FIG. 19. The remote device 6 can send the analyzed session report 106 to the physician's device 4, as shown by 107 in FIG. 19. The physician can review the analyzed session report 106 and produce a new physician's therapy report, if desired. If the physician produces a new physician's therapy report, the physician's device 4 can send the new physician's therapy report 109 to the remote device 6, as shown by 110 in FIG. 20. The remote device 6 can send urgent alerts to the physician's device (i.e., including portable phones, pagers, facsimile machines, e-mail accounts), for example, by text messaging, fax, e-mail, paging or combinations thereof. The remote device 6 can send and store the new physician's therapy report 109 in the database 10, as shown by 112 in FIG. 20.

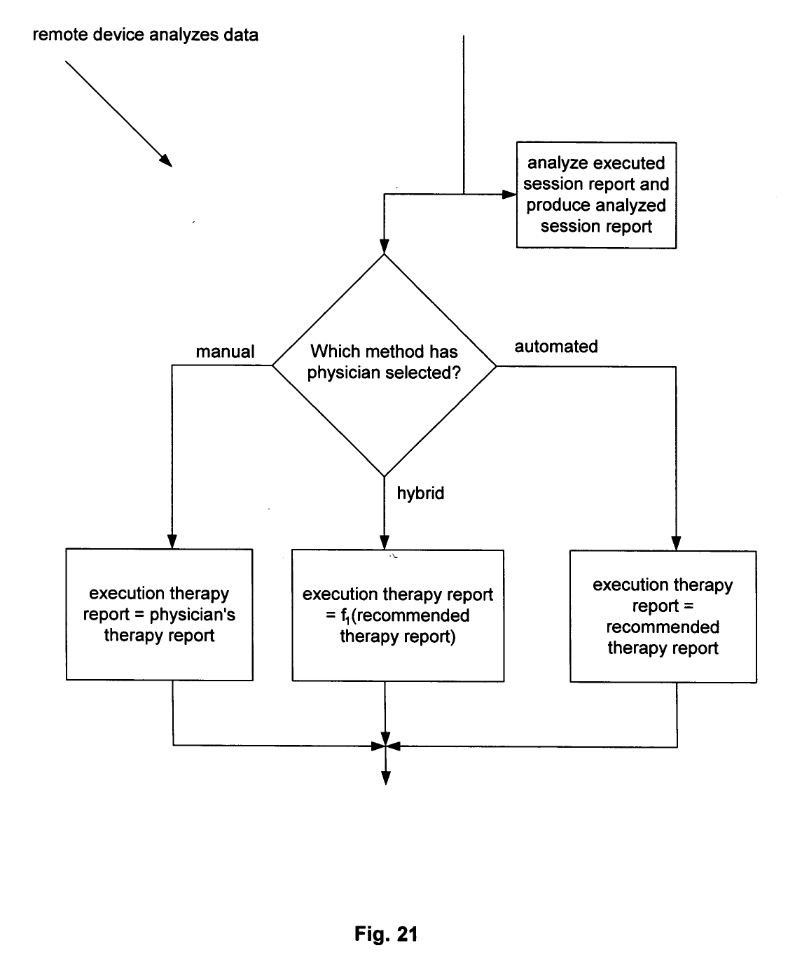

FIG. 21 illustrates analyzing the session report and the recommended and physician's therapy reports and producing the analyzed session report 106 and the execution therapy report, as shown by 103 in FIG. 16. The executed session report can be analyzed and an analyzed session report can be produced, as described supra. The execution therapy report can be produced as described supra, for example, in FIG. 11.

An Application Service Provider (ASP) can be used in conjunction with the system and/or method. The ASP can enable any of the devices 4, 6 and/or 8, the patient and/or the doctor, access over the Internet (e.g., by any of the devices 4, 6 and/or 8) or by telephone to applications and related services regarding the system 2 and use thereof. For example, the ASP can perform or assist in performing the sensory threshold test. In another example, the ASP can include a forum where patients can pose questions or other comments to trained professionals and/or other patients. In yet another example, the ASP can monitor and analyze the database 10, and the ASP can make suggestions therefrom to physicians and/or health monitoring organizations.

Methods and parts of methods are disclosed herein as being performed on one device 4, 6 and/or 8 for exemplary purposes only. As understood by one having ordinary skill in the art with this disclosure, any method or part of a method can be performed on any device 4, 6 and/or 8.

It is apparent to one skilled in the art that various changes and modifications can be made to this disclosure, and equivalents employed, without departing from the spirit and scope of the invention. Elements of systems, devices and methods shown with any embodiment are exemplary for the specific embodiment and can be used on other embodiments within this disclosure.

1. A system for applying sound to a neurological process amenable to treatment or augmentation by sound, the system comprising:
a first device, and
a second device communicating over a network to the first device, wherein the second device is configured to evaluate and treat the neurological deficiency.

2. The system of claim 1, wherein the second device treats the neurological deficiency according to a treatment protocol.

3. The system of claim 2, wherein the first device sends a treatment protocol to the second device.

4. The system of claim 3, further comprising a database readable by the first device, wherein the database comprises data regarding the neurological deficiency.

5. The system of claim 4, wherein the first device computes the treatment protocol with a computation, and wherein the computation comprises the data.

6. The system of claim 3, further comprising a third device communicating over a network to the first device.

7. The system of claim 6, wherein the third device can control the treatment protocol.

8. A system for applying sound to a neurological process amenable to treatment or augmentation by sound, the system comprising:
a first device configured to access a database comprising data, and
a second device configured to network with the first device, wherein the second device is configured to create therapy tones, and wherein the therapy tones are determined from a function comprising the data.

9. A first device for applying sound to a neurological process amenable to treatment or augmentation by sound, the system comprising, the device comprising:
a data transfer device, wherein the data transfer device is configured to communicate with a second device, and
an acoustic transducer,
wherein the data transfer device receives the therapeutic protocol from the second device.

10. A device for delivering sound to a user, the device comprising:
an acoustic transducer configured to deliver the sound,
a biometric sensor configured to receive feedback from the user.

11. The device of claim 10, further comprising an earpiece.

12. The device of claim 11, wherein the biometric sensor comprises an electric sensor

13. The device of claim 11, Wherein the biometric sensor comprises an optical sensor.

14. The device of claim 11, Wherein the biometric sensor comprises a pressure sensor.

15. The device of claim 11, further comprising an ambient sensor.

16. The device of claim 11, further comprising an ambient channel.

17. A device for transmission and reception of signals, comprising:
an earpiece comprising an acoustic transducer and an ambient sensor.

18. The device of claim 17, further comprising an ambient channel.

19. A method for applying sound to a neurological process amenable to treatment or augmentation by sound, the method comprising:
transferring a treatment protocol from a remote device to a local device comprising an acoustic transducer, and
performing the treatment protocol on the local device.

20. The method of claim 19, wherein the treatment protocol is computed by the remote device.

21. The method of claim 19, wherein the treatment protocol is the result of a computation comprising data from a database.

22. The method of claim 19, wherein the treatment protocol comprises a tone.

23. The method of claim 19, wherein the treatment protocol comprises an amplitude.

24. The method of claim 19, wherein the treatment protocol comprises a schedule.

25. A method for applying sound to a neurological process amenable to treatment or augmentation by sound, the method comprising:
applying therapy for the symptoms using a portable device, and
diagnosing the symptoms using the portable device.

26. The method of claim 25, wherein the portable device weighs less than about 2 pounds.

27. The method of claim 26, wherein the portable device weighs less than about 1 pound.

28. The method of claim 25, wherein diagnosing comprises receiving user feedback during therapy.

29. The method of claim 28, wherein user feedback comprises a biometric.

30. The method of claim 28, wherein user feedback comprises a qualitative user response.

31. The method of claim 25, wherein applying therapy comprises broadcasting a tone.

32. A system for applying sound to a neurological process amenable to treatment or augmentation by sound, the system comprising:
a first device configured to emit a first sound to a user, and wherein the first device senses electrical signals of the user's brain.

33. The system of claim 32, wherein the electrical signals are used to determine a second sound to be emitted to the user.

34. A method for applying sound to a neurological process for a user, wherein the neurological process is amenable to treatment or augmentation by sound, the method comprising:
performing a sensory threshold test with a first device on the user, wherein the sensory threshold test produces results, and
applying sound with the first device, wherein the sound is based on the results of the sensory threshold test.

35. A method for applying sound to a neurological process for a user, wherein the neurological process is amenable to treatment or augmentation by sound, the method comprising:
emitting sound with a first device to the user,
sensing a biometric from the user,
displaying the biometric to the user.

36. The method of claim 35, wherein the biometric comprises a brain signal.

37. The method of claim 35, wherein the biometric comprises a heart rate.

38. The method of claim 35, wherein displaying comprises substantially constant display of the biometric to the user.

39. A system for applying sound to a neurological process amenable to treatment or augmentation by sound, the system comprising:
a first device configured to emit a first sound to a user, wherein the first device senses electrical signals of the user's brain, and wherein the electrical signals are used to determine a second sound to be emitted to the user.

40. A method for applying sound to a neurological process for a user, wherein the neurological process is amenable to treatment or augmentation by sound, the method comprising:
emitting sound to the user,
retrieving biometric data from the user, and
coupling temporal data with the biometric data.

41. The method of claim 40, wherein the retrieving comprises retrieving by a first device, and wherein coupling comprises coupling by the first device.

42. A system for applying sound to a neurological process amenable to treatment or augmentation by sound, the system comprising:
a first device configured to emit a first sound to a user, wherein the first device comprises a sound generating processing unit.

43. The system of claim 42, wherein the sound generating processing unit is configured for efficient power consumption.

44. The system of claim 42, wherein the first device comprises more than one processing unit, and wherein the sound generating processing unit is configured to consume less power than the other processing units.

45. A method for applying sound to a neurological process for a user, wherein the neurological process is amenable to treatment or augmentation by sound, the method comprising:
generating sound, wherein generating comprises sound generating processing unit, and
emitting sound to the user.

46. The method of claim 45, wherein the sound generating processing unit is configured for efficient power consumption.
